(19) United States
(12) Patent Application Publication
(10) Pub. No.: US 2026/0325437 A1
(43) Pub. Date: Oct. 22, 2026

(54) COMPOSITION COMPRISING CORAL MUCUS, AND USE THEREOF

(71) Applicant: Inès GALTIER D’AURIAC, Saint Didier Au Mont D’or (FR)

(72) Inventor: Inès GALTIER D’AURIAC, Saint Didier Au Mont D’or (FR)

(21) Appl. No.: 19/107,757

(22) PCT Filed: Aug. 31, 2023
(86) PCT No.: PCT/EP2023/073961

§ 371 Date: Feb. 28, 2025

(30) Foreign Application Priority Data
Sep. 1, 2022 (FR) FR2208804

(51) Int. Cl. A61K 8/98

(52) U.S. Cl. CPC A61K 8/987; A61K 2800/91; A61K 2800/92

(57) ABSTRACT

One or more embodiments relate to a composition including mucus from at least one coral and use thereof in a cosmetic composition or a food supplement, a composition including coral mucus for therapeutic applications as well as a method for obtaining mucus from at least one coral including a step of retrieving mucus by suction or by gravity.

One or more embodiments relate to the field of natural products for the cosmetic industry, food supplement or pharmaceutical sector.

One or more embodiments relate more particularly to the use of coral mucus in these fields and more specifically for the formulation of cosmetic compositions or natural food supplements.

### TECHNICAL FIELD

The present invention relates to the field of natural products for the cosmetic industry, or wellness sector.

The field of the invention relates more particularly to the use of coral mucus in these fields and more specifically for the formulation of therapeutic or cosmetic compositions or natural food supplements.

### PRIOR ART

Corals are organisms with an as yet relatively little-known cellular and molecular physiology, and which live individually for up to several decades. They survive over long periods, despite being constantly exposed to bacterial, viral, fungal attacks, but also to climatic or pollution-related environmental risks; numerous parasitic organisms or opportunistic or specialist predators also represent a hazard. Therefore, the resistance of corals against all of these harmful effects suggests a potential source of novel natural molecules having advantageous effects in the field of cosmetics, dermatology, wellness or pharmaceuticals.

Mucus is a usually viscous substance created by living organisms. This superficial layer covering the skin of numerous marine animals is a protective barrier against external threats.

Coral particularly produces mucus. The ectoderm of corals secretes mucus. Mucus is defined as a mixture of proteins and carbohydrates. It forms a very fine mesh and could be used in the same way as a ‘fishing net’. Indeed, it will be able to trap food particles and elements from zooplancton. Once caught in this net, these elements will be taken to the mouth by ciliary movements or by tentacles.

Corals are currently highly monitored in relation to climate changes resulting in their bleaching and the reduction of their population.

Document KR 20140066521 relating to fish and the use of their mucus is known. Fish are aquatic vertebrate animals which are substantially different from corals which are invertebrate animals. The publication XP35127195 ‘Antibacterial activity of pseudoalterominas in the Coral Holobiont’ looks at the antibacterial effect of a family of Pseudoalterominas bacteria identified in the mucus of Platygyra sp, Porites sp, Stylophora sp. and Pocillopora sp corals and concludes that this family of bacterial should play a protective role in respect of corals against gram-positive bacteria that are pathogenic for corals.

The publication XP93038660 ‘Coral Mucus is a hot spot for viral infection’ relates to the evaluation of pathogenic virus spread in corals and concluded that coral mucus represents a highly favourable habitat for viral proliferation.

Document US2018/318205 for its part describes the use of red algae in cosmetics. Despite being known as ‘slender beaded coral weed’, red algae are plants and are not corals.

The aim of the present invention is that of providing a composition comprising coral mucus and the use thereof.

Further objects, features and advantages of the present invention will become apparent upon examining the following description and the appended drawings. It is understood that other advantages may be incorporated.

### SUMMARY

To achieve this objective, the invention relates, according to one embodiment, to a composition comprising, advantageously all or part of the, mucus from at least one coral, and advantageously an excipient or vehicle adapted for a subcutaneous application or for an oral administration or topical administration.

According to another aspect, the invention relates to a food supplement comprising mucus from at least one coral or comprising the composition comprising mucus from at least one coral.

According to another aspect, the invention relates to a cosmetic composition comprising mucus from at least one coral.

According to another aspect, the invention relates to the cosmetic use of coral mucus, advantageously of all or part of the coral mucus.

According to another aspect, the invention relates to the use of the cosmetic composition or food supplement.

According to another aspect, the invention relates to a composition comprising mucus from at least one coral intended for a therapeutic application.

According to another aspect, the invention relates to a method for obtaining mucus from at least one coral.

### DETAILED DESCRIPTION

Before starting a detailed review of embodiments of the invention, optional features are stated hereinafter, which can possibly be used in combination or alternatively:

According to one example, the composition comprises mucus from at least one coral.

According to one example, the at least one coral is chosen from soft mucus-producing corals or hard mucus-producing corals.

According to one example, the mucus, advantageously all or part, is in a viscous state or in a dry state.

According to one aspect, the invention relates to a cosmetic and non-therapeutic composition comprising mucus from at least one coral.

According to one example, the cosmetic and non-therapeutic composition is configured to be in the form of a shampoo, a mask, a soap, a gel, a cream, a lotion, a balm, a spray, a toothpaste. According to one example, the cosmetic use of the coral mucus, advantageously of all or part of the coral mucus, is in a form adapted for a topical application on the skin, mucosa or skin appendages.

According to one example, the cosmetic use of the coral mucus, advantageously of all or part of the coral mucus, is in a form adapted in the form of a shampoo, for example liquid or solid, a soap, a gel, a cream, a lotion, a balm, a spray, a mask, a toothpaste for example with soft or hard paste.

According to one aspect, the invention relates to a body hygiene product or a cosmetic care product comprising coral mucus, advantageously all or part of the coral mucus.

According to one aspect, the invention relates to a shampoo, a soap, a gel, a cream, a lotion, a balm, a spray, a mask, a toothpaste comprising coral mucus, advantageously all or part of the coral mucus.

According to one aspect, the invention relates to the use of the cosmetic and non-therapeutic composition for:
moisturising the skin or mucosa or skin appendages,
combating skin ageing,
combating skin imperfections, for example such as spots and excess sebum, for example such as acne,
reducing wrinkles and fine lines,
combating loss of skin elasticity,
combating loss of skin brightness, luminosity and glow,
reducing pore dilation,
reducing thickening of the superficial layer of the skin,
combating colour changes or pigmentation irregularities, abnormal and/or excessive keratinisation of the skin,
a thermoprotective effect,
skin barrier protection,
skin microbiota maintenance and protection,
cell renewal,
skin firmness.

According to one aspect, the invention relates to the use by topical application on the skin, mucosa or skin appendages of the cosmetic and non-therapeutic composition.

According to one aspect, the invention relates to a food supplement comprising the composition. According to one example, the food supplement is configured to be in the form of a dose, a hard capsule, a pastille, a tablet, a pill, a sachet of powder, ampoule of liquid, drop-dispensing bottle.

According to one aspect, the invention relates to the use of the food supplement for
moisturising the skin or mucosa or skin appendages,
combating skin ageing,
reducing wrinkles and fine lines,
combating loss of skin elasticity,
combating loss of skin brightness, luminosity and glow,
reducing pore dilation,
reducing thickening of the superficial layer of the skin,
combating colour changes or pigmentation irregularities, abnormal and/or excessive keratinisation of the skin,
skin barrier protection,
gut or skin microbiota maintenance and protection,
Improving intestinal and digestive comfort,
Promoting weight loss,
Reducing acidity, heartburn, heaviness, constipation or bloating,
Improving, maintaining and protecting gut microbiota,
Promoting cell renewal,
Skin firmness.

According to one aspect, the invention relates to the use of the food supplement by oral application of the food supplement.

According to one aspect, the invention relates to a composition intended for a therapeutic application for:
An anti-inflammatory effect
An antibacterial effect
An antiviral effect
An antioxidant effect,
The treatment of acne,
The treatment of eczema,
Skin photoprotection,

According to one aspect, the invention relates to a method for obtaining mucus from at least one coral comprising a step of retrieving mucus by suction or by gravity.

A parameter ‘substantially equal to/greater than/less than’ or ‘in the region of’ a given value means that this parameter is equal to/greater than/less than the given value, within a 10% margin, or within a 5% margin, of this value.

‘Mucus’ means a gelatinous, watery or viscous liquid. Coral mucus is a complex mixture of organic matter mainly composed of carbohydrates (mainly polysaccharides) and proteins, but also of fats, which is produced by the mucocytes of the epidermal coral layer of the ectoderm on the coral surface.

‘Coral’ means a living organism of the Anthozoa class (within the Cnidaria phylum), characterised by a calcareous (hard) or protein (soft) exoskeleton. Corals are invertebrate animals.

‘Coral mucus’ or ‘mucus from at least one coral’ means mucus or a mucus mixture. Preferably, the coral mucus according to the invention is either in the raw state, i.e. unmodified following its removal, except for a sterilisation treatment, in this case, the mucus is in a more or less liquid state commonly known as viscous state, or in a dried or freeze-dried state, i.e. unmodified except for its dehydration, the mucus is then preferably in a powder state. Coral mucus is also understood to mean in a solid form below its solidification temperature. For example, the invention applies to frozen coral mucus.

‘Anti-ageing’ means an effect intended to combat superficial ageing of the skin such as for example slackness, sagging, wrinkles and fine lines, dryness, dehydration, loss of skin elasticity, loss of skin brightness, luminosity and glow, pore dilation, thickening of the superficial layer, colour changes or pigmentation irregularities, abnormal and/or excessive keratinisation of the skin.

‘Moisturiser’ means a product that moisturises the skin and the superficial layer of the epidermis and prevent its dehydration by rebuilding the hydrolipidic layer, natural protection of the skin and all the ensuing properties.

‘UV protection’ or ‘photoprotection’ means the various means capable of countering skin damage induced by solar radiation.

‘Solar radiation’ means the radiation emitted by the sun and reaching the Earth's surface in particular UV-A and UV-B rays, but also infrared rays causing heating of the skin.

‘Probiotic’ effect means protection of the skin or gut microbiota (or flora) from external attacks. When pathogenic bacteria try to become established, probiotics, which are microscopic living organisms (yeasts, viruses, bacteria, etc.), produce toxic substances to destroy them.

A ‘thermoprotective effect’ means, for hair cosmetics, protection against heat caused by a hair-dryer or a curling iron.

‘Cosmetically acceptable’ or ‘adapted for cosmetic use’ means compatible with the skin and/or mucosa and/or skin appendages, having a pleasant colour, odour or texture and not giving rise to unacceptable discomfort (stinging, tautness, redness), capable of deterring the consumer from using this composition.

‘Food supplement’ means a product as defined in Directive 2002/46/EC of the European Parliament and of the Council of 10 Jun. 2002 on the approximation of the laws of the Member States relating to food supplements. ‘Food supplement’ means a foodstuff the purpose of which is to supplement the normal diet and which is a concentrated source of nutrients or other substances with a nutritional or physiological effect, alone or in combination.

The invention relates to a composition comprising mucus from at least one coral.

The mucus is mucus from a single coral or a mixture of mucus from several corals.

According to the invention, the mucus is either in a viscous state, or in a dry state for example in powder form. The mucus is advantageously raw mucus, i.e. unmodified also known as native, except for a sterilisation and/or freeze-drying or dehydration treatment.

According to one possibility, the mucus is understood to mean all or part of the mucus. The mucus having been enriched or not. According to one possibility, the mucus comprises adapted concentrations of the different constituents.

According to one possibility of the invention, the at least one coral is chosen from hard corals from the Acropora family Acropora yongei, Porites, Hermatypic, Porites astreoides, Diploria strigosa, Montastrea annularis, Colpophyllia natans, Acropora palmata, Lobophyllia hemprichii, Fungia scutaria, Fungia repanda, Acropora danai, Pocillopora eydouxi, Pocillopora meandrian, Fungia fungites, Fungia repanda, Diaseris distorta, Fungia scutaria, Cycloseris costulata, Cycloseris doederleini, Cycloseris marginata, Fungia actiniformis, Fungia danai, Fungia echinata, Fungia fungites, Fungia granulosa, Fungia horridae, Fungia Klunzingeri, Fungia scruposa, Fungia sommmervillei, Platigyra sp., M. annularis, P. astreoides, F. scutaria, Palythoa sp., F. scutaria, Acropora spp., Montipora digitata, Euphyllia sp., Porites porites, Porites compressa, Acropora sp., Siderastrea siderea, Agaricia agaricites, Madracis mirabilis, Montastrea cavernosa, P. porites, Eusmilia fastigiata, Mussa angulosa, Favia fragum, Colpophyllia natans, D. strigosa, Cladocoral cespitosa, Mycetophyllia reesi.

According to another possibility of the invention, the at least one coral is chosen from soft corals chosen from Sarcophyton sp., Lemnalia sp., Cespitularia sp., Rumphella aggregata, Red Sea Coelenterata, Lobophyton sp, Rhytisma fulvum fulvum and Xenia sp.

According to one possibility, the composition consists only of mucus from at least one coral.

According to one embodiment, the composition comprises at least one excipient or one vehicle adapted for a subcutaneous application.

According to one embodiment, the composition comprises at least one excipient or vehicle adapted for a topical application.

According to one embodiment, the composition comprises at least one excipient or vehicle adapted for an oral administration.

According to one possibility, the composition comprises at least one cosmetically acceptable excipient or vehicle. The composition is a cosmetic, non-therapeutic composition, i.e. the cosmetic composition is a composition adapted for a cosmetic application.

The cosmetic composition is advantageously intended for a topical application. The cosmetic composition preferably has a form adapted for a topical or subcutaneous application. Preferably, the cosmetic composition is intended and advantageously adapted for an application on the skin including the lips, hair, eyelashes, eyebrows and/or nails. The cosmetic composition is for example in the form of a shampoo, a soap, a gel, a cream, a lotion, a balm, a spray.

The cosmetic composition is advantageously intended to be used for moisturising the skin, mucosa or skin appendages, combating skin ageing, reducing wrinkles and fine lines, combating loss of skin elasticity, combating loss of skin brightness, luminosity or glow, reducing pore dilation, reducing thickening of the superficial layer of the skin, combating colour changes or pigmentation irregularities, abnormal and/or excessive keratinisation of the skin, protecting the skin barrier, improving, maintaining and protecting skin microbiota, thermal protection of hair, promoting cell renewal in particular in the skin, improving skin firmness.

According to one possibility, the composition comprises at least one excipient or vehicle configured to be used as a food supplement. The invention relates, according to one aspect, to a food supplement comprising mucus from at least one coral or a composition comprising mucus from at least one coral as described hereinabove.

The food supplement is advantageously intended for an oral administration. The food supplement preferably has a form adapted for an oral administration such as for example a liquid or pseudo-liquid such as viscous, i.e. in the form of a syrup, gel, spray, ampoule, hard capsule, drop-dispensing bottle or such as for example in solid dose form such as a soft capsule, a hard capsule, a pastille, a tablet, a pill, a lozenge, a sachet of powder. Other similar forms of liquid or powder preparations intended to be taken in measured units can be used.

Advantageously, the food supplement is intended to be used in dose form.

According to one embodiment, the food supplement is intended to be used daily in dose form, from 1 to 3 times daily, for example in the morning, at noon and/or in the evening.

The food supplement is advantageously intended to be used for moisturising the skin, mucosa or skin appendages, combating skin ageing, reducing wrinkles and fine lines, combating loss of skin elasticity, combating loss of skin brightness, luminosity or glow, reducing pore dilation, reducing thickening of the superficial layer of the skin, combating colour changes or pigmentation irregularities, abnormal and/or excessive keratinisation of the skin, protecting the skin barrier, improving, maintaining and protecting gut or skin microbiota, improving intestinal and digestive comfort, promoting weight loss or reducing acidity, heartburn, heaviness, constipation or bloating. According to one possibility, the composition comprises at least one pharmaceutically acceptable excipient or vehicle. The composition is a therapeutic composition, i.e. the composition is a composition adapted for a therapeutic application.

To this end, the invention relates to a composition comprising mucus from at least one coral intended for a therapeutic application. The therapeutic composition is advantageously intended for a topical or subcutaneous or orally administered application. The therapeutic composition preferably has a form adapted for a topical or subcutaneous or oral application. Preferably, the therapeutic composition is intended and advantageously adapted for an application on the skin including the lips, hair, eyelashes, eyebrows and/or nails. The therapeutic composition is for example in the form of a shampoo, a soap, a gel, a cream, a lotion, a balm, a spray. The therapeutic composition is also adapted for an oral administration, being for example in the form of a syrup, gel, spray, ampoule, hard capsule, drop-dispensing bottle or such as for example in solid dose form such as a soft capsule, a hard capsule, a pastille, a tablet, a pill, a lozenge, a sachet of powder. The cosmetic composition is also adapted for a subcutaneous administration for example by injection.

The composition is advantageously intended to be used for a therapeutic application for an anti-inflammatory effect, an antibacterial effect, an antiviral effect, treatment of acne, treatment of eczema, skin photoprotection, an antioxidant effect, cell repair.

For example, the quantity of mucus in cosmetic applications is between 0.5% and 50% by weight of the total weight of the final composition.

For example, the quantity of mucus in therapeutic applications is between 50.5% and 100% by weight of the total weight of the final composition.

For example, the quantity of mucus in food supplement applications is between 0.5% and 100% by weight of the total weight of the final composition.

According to another aspect, the invention relates to a method for obtaining mucus from at least one coral. The method according to the invention comprises a step of retrieving mucus advantageously without damaging the coral. The coral is not destroyed, is not cut. The coral remains intact. The method according to the invention is non-invasive.

According to a preferred possibility, the retrieval step is carried out by suction of the mucus or by gravity.

More specifically, the coral is turned over to remove the mucus by gravity or the mucus can also be collected with a syringe by suction.

According to one possibility, the method comprises a step preceding the retrieval step which is a step of taking the coral out of the water.

According to one possibility, the method comprises a step after the step of taking the coral out of the water which is a step of removing excess water, more specifically the coral is sponged.

According to one possibility, the method comprises a step of stimulating mucus production before the retrieval step and preferably after the step of taking the coral out of the water and preferably after the step of removing the excess water. For example, the step of stimulating mucus production is carried out by contact with air and/or by stirring with a glass rod.

Advantageously, the method according to the invention comprises a step of sterilising the mucus. Preferably, the sterilisation step is carried out by exposing the mucus to UV radiation, for example a UV lamp, for a predefined time. This step makes it possible to remove micro-organisms, preferably viruses and parasites, from the mucus without degrading the primary structure of the mucus.

For example, sterilisation is carried out by exposing the mucus under a UV lamp for a predefined time for example in the region of two hours in an Eppendorf® type tube, or by gamma photon irradiation for a predefined time. According to one possibility, the tube is then closed and place in a paper cryobox. The whole box is then treated with gamma photons at a dose of 25 to 50 kGy by applying a standard commercial method.

Advantageously, the method according to the invention comprises a step of antibiotic treatment of the mucus. The mucus is treated with at least one antibiotic or several antibiotics selected to destroy certain bacteria and preserve others. This step is particularly useful in the case of use as a probiotic.

Preferably, the cosmetic composition and/or the food supplement are formulated from natural ingredients.

According to one possibility, the invention relates to a method comprising the cosmetic application of coral mucus, advantageously all or part of the coral mucus. Preferably, the cosmetic application comprises a topical application. Preferably, the cosmetic application comprises application on the skin including the lips, hair, eyelashes, eyebrows and/or nails. According to one possibility, the method comprises the cosmetic application of a composition comprising coral mucus.

### Example 1—Cosmetic Composition for a Cream, a Lotion or a Gel

Coral mucus, water, organic glycerine, organic Butyrospermum Parkii (shea butter), organic Coco Nucifera (coconut) oil, organic Simmondsia Chinensis (Jojoba) seed oil, cetearyl olivate, sorbitan olivate, Cassia Angustifolia seed polysacchardie, Alteromonas ferment extract, jojoba esters, seed wax, polyglycerin-3, beta-glucan, algin, xanthan gum, organic Cedrus Atlantica (cedarwood) essential oil and salvia officinalis (sage) oil, vitamin E.

### Example 2—Cosmetic Composition for a Cream, a Lotion or a Gel

Coral mucus, water, caprylic/capric triglyceride, squalane (olive), dipalmitoyl hydroxyproline, glycerine, cetearyl alcohol, cetearyl glucoside, Helianthus Annuus (sunflower seed) oil, alkyl glucoside, glyceryl stearate, stearic acid, Leuconostoc/Radish Root Ferment Filtrate, Hydroxyethylcellulose, Persea Gratissima (Avocado) oil, Simmondsia Chinensis (Jojoba) seed oil, Capryloyl Glycine, Undecylenoyl Glycine, Glycine, Calendula Officinalis flower extract.

### Example 3—Food Supplement in Daily-Dose Hard Capsule Form

Coral Mucus (280 mg for 1 hard capsule)-Anti-caking agent: vegetable magnesium stearate
Gelatine shell: natural chlorophyll

### Example 4—Method for Retrieving Mucus

The coral chosen is Acropora yongei. The coral is taken out of the aquarium. The coral is advantageously turned over and preferably exposed to air for a predefined time, for example 10 seconds advantageously to reduce coral stress. The mucus is then collected directly in a glass or Falcon® tube.

### Example 5—Method for Retrieving Mucus

The coral chosen is Acropora yongei. The coral is taken out of the aquarium. The coral is advantageously turned over and preferably exposed to air and mechanically stimulated for a predefined time, for example 5 seconds advantageously to reduce coral stress. Mechanical stimulation is carried out with a rod preferably made of glass that is skimmed along the coral. The mucus is then collected directly in a glass or Falcon® tube.

### Example 6—Method for Retrieving Mucus

The coral chosen is Acropora yongei. The coral is kept inside the aquarium. The coral is mechanically stimulated for a predefined time, for example 5 seconds advantageously to reduce coral stress. Mechanical stimulation is carried out with a rod preferably made of glass that is skimmed along the coral or with water jets. The mucus is then collected with a pipette.

The invention is not limited to the previously-described embodiments and encompasses all of the embodiments covered by the invention.

1. A composition comprising mucus from at least one coral.

2. The composition according to claim 1 comprising at least one excipient or vehicle adapted for a topical application.

3. The composition according to claim 1 comprising at least one excipient or vehicle adapted for a subcutaneous application.

4. The composition according to claim 1 comprising at least one excipient or vehicle adapted for an oral administration.

5. The composition according to claims 1, wherein the at least one coral is chosen from soft mucus-producing corals or hard mucus-producing corals.

6. The composition according to claims 1, wherein the mucus is in a viscous state or in a dry state.

7. A method of using mucus from at least one coral comprising applying mucus from at least one coral as all or part of a cosmetic and non-therapeutic composition.

8. A method of usinge the composition according to claim 1 comprising applying the composition in a form adapted for a topical application, on the skin, mucosa or skin appendages.

9. The method according to claim 8 comprising applying the composition, in the form of a shampoo, a soap, a gel, a cream, a lotion, a balm, a spray, a toothpaste.

10. A food supplement comprising the composition according to claim.

11. The food supplement according to claim 10 wherein the food supplement is in a form of a dose, a hard capsule, a pastille, a tablet, a pill, a sachet of powder, an ampoule of liquid, or a drop-dispensing bottle.

12. The food supplement according to claim 10 wherein the food supplement is in a form adapted for an oral application.

13. A method of using the composition according to claim 1 comprising administration to a patient for a therapeutic application for:
an anti-inflammatory effect, and/or
an antiviral effect, and/or
an antioxidant effect, and/or
a treatment for acne, and/or
a treatment for eczema, and/or
skin photoprotection
